(19) United States
(12) Patent Application Publication
(10) Pub. No.: US 2011/0293534 A1
(43) Pub. Date: Dec. 1, 2011

(54) Aerosol can spray incorporating a nutritional cell scaffold for use as first aid treatment for wounds/burns and for use in cell therapy/tissue repair

(76) Inventor: Denis Eon Solomon, Wigan (GB)

(21) Appl. No.: 13/068,967

(22) Filed: May 25, 2011

Related U.S. Application Data
(60) Provisional application No. 61/397,494, filed on Jun. 14, 2010.

(30) Foreign Application Priority Data
Jun. 16, 2009 (GB) GB 0910.314.4

(51) Int. Cl. A61K 9/12; A61Q 19/00; A61P 17/02; A61K 38/12; A61K 31/702

(52) U.S. Cl. 424/45; 514/2.4; 514/54

(57) ABSTRACT

A First Aid aerosol can spray using a propellant authorized for human use. Spray will be in the form of a ‘light emulsion’ for use on burns and wounds at incident sites. If propellant is not used, two of the spray constituents, namely sterile cornstarch granules, a nutritional cell scaffold and antibiotic(s) may be used for guided subcutaneous tissue repair. Specific cell culture techniques are described for both epidermal and dermal cells which are to be externally applied to aid wound healing of wider, deeper wounds/burns in terms of total body area affected.

### FIELD OF INVENTION

This specification relates to First Aid and guided cellular repair with regards to wounds and burns.

### RELATED UK APPLICATION DATA

A form of this specification, GB 0910314.4 was filed on 16 Jun. 2009 and published by the UK Patent Gazette on Dec. 22, 2010.

Ownership

This specification did not involve any sponsorship by anyone. It is based on the author's own experience in and outside of a tissue culture laboratory.

### BACKGROUND

Current medical practice consists of an ‘Outside-in’ wound/burn treatment at present. There is no nutrition applied to damaged subcutaneous tissue on application of a skin graft or cultured epithelial autograft (CEA), essentially ‘sheet’ materials. Without initial adherence, plasmatic imbibition, and revascularization, the graft will not survive. Plasmatic imbibition, a form of passive diffusion furnishes the only source of cellular repair nourishment.

In other words, damaged tissue is further burdened by the overlay of a skin graft and is charged with the supply of the necessary cellular nourishment for subcutaneous tissue repair. The same may be said about all cultured skin substitutes e.g. Apligraf™ used as an overlay on the wound bed. Immobilization must be practised to allow the skin graft to ‘take’ and the ‘donor site’ of the skin graft must also be medically treated.

There are several factors that could affect the skin graft from ‘taking’. These are infection, haematoma or seroma formation and shearing forces. Meshing helps to minimise the risk of failure due to haematoma formation as it allows drainage of blood and body fluids. Healing occurs as the spaces between the mesh fill in with new epithelial skin growth. The disadvantages of meshing are that it is a less durable graft than a sheet graft and that the larger the mesh, the greater the permanent scarring. In instances of skin graft overlay, there is no involvement of the upper most layer of the dermis (the papillary dermis) to wound repair/tissue regeneration, being left relatively intact on application.

E. Mansilla et al. in their 2001 article on the Derma Project in Argentina suggested that ideal skin substitutes would have the following characteristics: unlimited amounts; ready to be implanted; simple inexpensive manufacture; non-immunogenic; permanently accepted by the recipient; sterile. No mention of a biodegradable cell scaffold with nutritional value. For a review of currently available skin substitutes, see Y M Bello & A F Falabella in Dermatology Clinics (2001), 2(5): 305-13.

Seepage from wounds and burns before proper hospital care can be obtained is of great clinical concern. Maintaining homeostasis in burns patients is a major medical problem even after admission to hospital. Any product that can alleviate seepage of body fluids, stems bleeding and provides pain-relieving properties will be of tremendous use in an emergency situation. USA medical ambulances do not stock an easy-carry can that is leak-proof and sterile for use at incident sites. These can be manhandled by paramedics without puncture of can, spillage or risk from outside infection of contents. Burn patients are simply covered with a wet blanket. Soldiers in the field of battle are left with seeping wounds and burns until helicopter evacuation.

Why use cornstarch? It is a natural product extracted from shells of corn; a white granular carbohydrate occurring in the ectoderm of corn kernel. It enjoys wide use in talcum powder, in domestic cooking as a thickener (a binding agent) for soups and stews and as a domestic and commercial laundry product. The product has been around for over a century without any complaints of toxicity when used on the human body for the relief of itchy or irritated skin, e.g. rashes, hives or poison ivy, prickly heat and as a means of soothing sunburn and diaper rash. A cornstarch granule under the microscope has the morphology of a donut. Aggregates of cornstarch occur in solution or in a cell culture medium allowing multi-directional cell attachment. Type of cell could vary. Wound fluid will determine type and size of aggregates occurring.

Dr Denis Solomon was burned on the first web space of his right hand (upper surface) through accidental contact with a preheated (375° F.) domestic oven filament. A second-degree burn i.e. raised epidermis plus bleeding resulted. No scar tissue or darkening of the burn area resulted after concomitant repeated use of a binding agent, sterile cornstarch in granule form, overlaid with an over-the-counter triple antibiotic ointment (Bacitracin Zinc, 400 units, Neomycin Sulphate, 3.5 mg Polymyxin B Sulfate, 5000 units) sold at a local Miami, Fla. drugstore. A simple gauze dressing was used. It was observed that there was air drying of the top layer of the resulting white ‘sticky powdery mixture’. Today, it is unlikely a trained clinical observer could tell that a burn or other subcutaneous injury occurred. Application of the cornstarch granules constituted a microscopic scaffold and the added triple antibiotic led to scientific observations and conclusions used in the development of said aerosol can spray.

Particle size was a consideration (see www.engineeringtoolbox.com). Red blood cells are 5-10 microns in size, whereas cornstarch is of the order of 0.1-0.8 microns, but the latter do form aggregates of different sizes. Published reports of the invasive use of cornstarch granules as a lone tool in wound healing or as glove powder inadvertently contaminating wound beds resulted in tissue inflammation. A Medline search for published material containing the concomitant use of cornstarch granules and an antibiotic (or antibiotics) and/or clove oil yielded no results. Clove oil (oil of Cloves) is well known and in widespread use in dentistry as a pain reliever.

An Inside-Out Technique

Medical management of subcutaneous wound healing has not been practised by placing a quantity of a microscopic absorbent scaffold composed of a nutritional binding agent together with an antibiotic ointment within the wound bed to allow epithelial-mesenchymal crosstalk to dictate the architecture and regeneration of damaged tissue at its own pace. (Alternatively, the antibiotic(s) may be substituted with a natural compound, clove oil (oil of Cloves), with credited pain-relieving properties). Writing a Scientific American article (March 2008) ‘Regrowing limbs: Can people regenerate body parts?’ Prof. Ken Muneoka makes the point that medical treatment inhibits tissue regeneration. In other words, since the 1800s, physicians have been installing a default repair mechanism, overloading the wound bed, resulting in scar tissue while causing the suppression of the regenerative mechanism leading to tissue repair.

In essence, the microscopic scaffold sets up the subsequential evolution of its own three-dimensional coordinates within the wound bed. Wound seepage will cause the manifestation of the binding, aggregation and absorbent properties of the scaffold. Body heat will cause the antibiotic ointment to degrade to a semi-liquid form and bond to the scaffold resulting in something approaching a ‘light emulsion’. Cells within the damaged subcutaneous tissue will be directly fed by the carbohydrate nutritional agent, which is of course, biodegradable. Repeated application of this ‘light emulsion’ will cause the normal and natural regeneration of both the epidermis and dermis. The emulsion can be manufactured as a First Aid aerosol can spray with an authorized propellant for human use, currently being used without contra-indications in bronchial inhalants.

The primary scaffold of wound healing is the fibrin blood clot and the cascade of subsequent events results in scar tissue. Interference with the formation and amount of the fibrin clot by application of the above mentioned emulsion does cause subcutaneous tissue repair with little or no scar tissue. This is where the scientific study of tissue regeneration and drawn conclusions clashes with traditional medical practice.

Two ‘case reports’ described below will serve to illustrate this conclusion:

(1) An 88 year-old man had undergone Mohs surgery for skin cancer on his right cheek. Post-operatively, he was medically advised to use Vaseline and hydrogen peroxide for home care wound management. Seepage resulted and the skin had the appearance of a very severe abrasion. Within 6 weeks, with use of the ‘sticky powdery mixture’ (cornstarch granules and triple antibiotic ointment) applied after every two days with a change of normal gauze dressing on home premises, the dermis was rebuilt flush with his cheek and only close examination would reveal two thin scalpel lines. There was no skin crater and no scar tissue. The ointment was layered over the scaffold/binding agent.

(2)An FDA-approved commercial laboratory produced sample aerosol spray cans containing clove oil (oil of Cloves; 12% v/v) and sterile cornstarch granules. A Confidential Disclosure Agreement was signed. Outside office hours and at a private residence, a staff researcher (Department of Dermatology & Cutaneous Surgery, University of Miami School of Medicine) used a sample can of the First Aid spray at time of incident on a bleeding gash on his knee. Blood clotting was aided and a measure of pain relief was experienced. The gash healed normally without tissue inflammation and there was no scar tissue.

My in vitro finding that both papillary and reticular dermal fibroblasts and their secreted mixed extracellular matrix (ECM) plus a full complement of epidermal cells (not only keratinocytes)) are required for the in vitro ‘autoengineering’ of a multilayered cellular island. This multilayered structure represents the precursor of the epidermal ‘brown rosette’, a consequence of overnight Dispase digestion of human epidermis (See Solomon D E (2002) in Int. J. Exp. Path. vol. 83, p 209-216 and UK patent applications 2006, 2007).

In the light of these findings, perhaps it is necessary to spray on the subcutaneous wound bed in orderly fashion: the nutritional binding agent scaffold plus antibiotic followed by a mixture of autologous papillary and reticular dermal fibroblasts repeating until there is wound coverage. The upper layers of the emulsion containing scaffold plus antibiotic (and/or clove oil) will become air dried and impermeable while the lower layers in contact with the wound bed and body heat will remain semi-soluble. Coverage with a normal gauze dressing is required. Later on, autologous brown rosettes of epidermal cells may be added to complete the required cellular content and structure of the repaired skin.

Classification

Worldwide search of classified patent documents designates this specification in the following areas of IPC as: A61K, A61P.

### RELATED US APPLICATION DATA

This specification was filed on Jun. 14, 2010 with application No. 61/397, 494 as a provisional ‘small entity’ patent application. This date is being claimed as a priority date. With this full filing of the specification under ‘small entity’ provision, the previous provisional filing is being discontinued.

### PRIOR ART

Prior art include Topical anti-microbial compositions by De Olivera et al., U.S. application Ser. No. 11/238021, publication number US 2007/0071705 A1, Mar. 29, 2007. Therein lies no mention of subcutaneous wound healing; only of ‘restoring and repairing lipid barrier of skin’, a focus on tapioca starch with no mention of use as a cell scaffold in guided tissue regeneration; speaks to ‘indirect’ application to skin to treat skin infections.

Antibiotic compositions containing Novobiocin, by Merck and Company; application number UK 30999/56, Oct. 11, 1956, patent number 824,785; A61k, publication date: Dec. 2, 1959. Therein lies a vague mention of application to wounds; no mention of a First Aid product to be used at time of incident; grinding/pulverizing powder is mentioned which would not result in the cornstarch granules remaining intact as in my specification for their intended use as nutritional cell scaffold; nor is there mention of guided skin tissue regeneration. When the percentage of cornstarch and cornstarch paste is calculated, they amount to only 6% of mixture listed in Example 1 lines 55-60.

6% of cornstarch (no intact granules mentioned) would be insufficient for the purposes listed in my specification. Additionally, in line 43, it states ‘these compositions can be prepared in accordance with conventional practices known in the art’. Late 1950s technology is reflected in the use of corn starch as a 10% paste in Example 2, lines 65-75 where the calculated amount of dry cornstarch is 1%; totally insufficient for the purposes in my specification.

There is also the author's (D. E. Solomon) own granted 2004 UK patent, GB 2365443A entitled: ‘A surgical-medical dressing for the treatment of body burns and for wound healing’ filed 4 Apr. 2000; granted 22 Dec. 2004. Cornstarch powder is mentioned in claim 5, not pure, sterile cornstarch granules as in this specification. Its use here as a nutritional cell scaffold for epidermal and dermal cells is not mentioned, neither is guided tissue regeneration.

There is no prior art for a nutritional cell scaffold for use in skin tissue repair, after injury. In the related patents quoted above, there are no relevant granted Claims.

### SUMMARY OF THE PRODUCT (‘THE INVENTION’)

A can with a suitable nozzle to facilitate an aerosol emulsion spray will be required. The aerosol propellant will be environmentally safe and of the kind already in use in bronchial inhalants. A emulsion of pure, sterile cornstarch (previously subjected to toxicology tests) will be mixed with the antibiotic and/or the oil of cloves and the aerosol propellant to provide the can fill. The aerosol propellant does not compromise the structural integrity of the cornstarch granules. The final spray will be subjected to chemical toxicity and allergy tests. Without the propellant, two of three spray constituents, may be used for guided tissue repair by themselves. Using specified cell culture techniques, described herein will aid coverage of deeper and wider wounds/burns (in terms of damaged body area).

### DETAILED DESCRIPTION OF INVENTION

An aerosol can spray composed of two or three constituents and an aerosol propellant authorized for human use. This propellant will be environmentally safe of the kind already in use in bronchial inhalants. Said constituents can be: sterile cornstarch granules and antibiotics or cornstarch granules and clove oil (Oil of cloves) or a mixture of all three, namely pure cornstarch granules, antibiotics and clove oil to provide a pain relieving spray.

Antibiotics may include, just for example: Bacitracin Zinc, Neomycin Sulphate and Polymyxin B Sulfate. Up to 12% (v/v) of clove oil may be safely used. Constituents will be contained in a leak proof can with a nozzle for easy dispensation of spray. Chemical toxicity and allergy tests will be performed on all three ingredients and on each batch of finished product. The spray will be in the form of a ‘light emulsion’ providing a temporary ‘occlusive dressing’ to wounds and burns at incident sites.

Manufacture will be by an appropriately licensed manufacturer. It is envisaged that individual cans would have contents, by volume, of 200 to 600 milliliters. Additionally, the cornstarch granules/antibiotic(s) may be employed as a nutritional cellular scaffold for use in tissue repair, cell therapy and cosmetic applications (no propellant). Isolation of cells, epidermal and dermal, must be expedited by cell culture techniques as described in this specification, to be applied to a wider damaged body area.

Cellular Therapy/Tissue Repair Using Specified Cell Culture Techniques.

Dermal Fibroblasts:

After epidermis stripping, dermal fibroblasts, both papillary and reticular, may be obtained by mechanical scraping of the dermis. The cells are loosened from the scalpel blade by swirling in a centrifuge tube containing cell medium e.g. Medium 199 and 20% serum. The tube is covered and manually swirled. Cells are left to settle down for 20 minutes. The top portion is pipetted off, put in a plastic petri dish and incubated for cell growth observation. The bottom portion containing most cells is plated in another petri dish. In both instances the culture dishes containing an aliquot of complete culture medium are pre-warmed in the incubator. Within 24-36 hours the culture medium can be changed, because fibroblast attachment occurs fairly rapidly. If necessary later, 5 mM EDTA/PBS is used to pool cells into the petri dish containing the aforementioned bottom portion of cells. This technique circumvents the use of enzymes and centrifugation and will isolate a truly representative cellular population of both papillary and reticular fibroblasts. Published reports to date use enzymatic means on minced dermis to obtain a yield of dermal fibroblasts. Fibroblasts are not affected by the previous overnight use of Dispase. They multiply with normal morphology within a normal time frame, produce a normal extracellular matrix and ‘passage’ (sub-culture) normally. Skin tissue samples may be ‘fresh’ or cadaver (within 24 hours of death).

It should be noted that the smallest piece of patient skin tissue will yield a population of autologous dermal fibroblasts which under sterile tissue culture conditions will quite easily multiply into millions of cells within weeks. The patient's own serum can be employed. Any number of nutritional culture media can be routinely employed e.g. Medium 199. Since the fibroblasts release their own growth factors, this cellular population multiplication process if expedited by subculturing (passaging) with (Ethylene diamine tetra-acetic acid/phosphate buffered saline), EDTA/PBS pH 7.4 (no enzyme digestion, hence minimal cellular perturbation) to retract the cell layers (at confluence or semi-confluence) would be cost effective for repeat applications of dermal fibroblasts at dressing changes (See Solomon D E in IJEP 1992, 2002).

Keratinocytes:

In published reports, epidermis is stripped after enzymatic digestion with Trypsin or Thermolysin or Dispase. Then basal keratinocytes are carefully scraped off the uppermost surface of the dermis. There usually is fibroblast contamination. Delivery of keratinocytes using fibrin glue via an aerosol spray has been recorded (Currie et al 2003, Duncan et al 2005).

To neutralise Dispase, the enzyme manufacturers in their catalogues recommend using 5-10 mM EDTA (ethylene diamine tetra-acetic acid). To passage the primary culture of keratinocytes, researchers have subsequently employed Trypsin, commercially sold as a Trypsin-EDTA mixture. It should come as no surprise that the resulting keratinocytes are dysfunctional cells because of the double dose of EDTA. Also researchers have ignored the 1981 finding of Barton and Marks that trypsin affects the membranes of keratinocytes.

On the other hand, if the epidermis is digested for 18 hours with trypsin before stripping and trypsin again is used to passage primary keratinocytes, the cells will not be top quality. Double use of enzymes, one following another, should be avoided. It has not been realised that leaving the enzyme Dispase in contact with human epidermis on an overnight basis has two consequences. The enzyme will lose its efficacy, hence no need to use any neutralizing agent and furthermore, the human epidermis tissue samples disassemble into cellular structures christened as ‘brown rosettes’ by Solomon D E in Int. J. Exp. Path. (2002). These rosettes contain epidermal cells, not only keratinocytes. 20% serum and culture medium are described therein. Said author has performed this tissue culture procedure over five dozen times (n˜65).

It is being suggested here that the epidermal ‘brown rosettes’ which show no specific polarity of integrin receptors be applied to wounds/burns instead of a emulsion of keratinocytes in an aerosol spray. Fibroblast contamination will be totally avoided. The ‘brown rosettes’ can attach to an ECM substrate in an upright or inverted configuration. It is presumed that only basal brown' rosettes attach to a ‘mixed’ (papillary and reticular, obtained by scraping) dermal fibroblast (extracellular matrix) ECM (or HUVECs ECM) which has been experimentally observed on a routine basis by this author-see also Spichkina et al 2006, UK patent applications 2006, 2007-D E Solomon.

This idea is based on the report that although the conformation of the keratinocyte betal integrins differs between the basal and the lateral/apical membrane domains, there is no intrinsic polarity in the ligand binding potential of the receptors (Bishop et al 1998). This was underlined in my 2006 UK patent application, where although the speck of epidermis was inverted, the observed process of ‘autoengineering’ by unmanipulated epidermal and dermal cells was observed in a classic tissue culture ‘explant’ procedure. The inverted ‘brown rosettes’ do shed their content of basal epidermal cells including additional in situ cells e.g. melanocytes on an appropriate ECM substrate.

Scientific References:

The Derma Project: Present and future possibilities of skin procurement for the treatment of large burns in Argentina, Tissue Engineering and the Cadaver Skin Bank. Mansilla E, Arrúa J, Salas E, Gardiner C, Marchessi N, Manfredi D, Schreiner A, Mosquera R, Gil M A, Gardenal L, Ball Lima M, Marin G, Drago H, Sturla F, Menna M E, Sorratti C, Piccinelli G. in Transplant Proc. 2001 Feb-Mar; 33(1-2):637-9.

Regrowing human limbs. Muneoka K, Han M, Gardiner D M. in Sci Am. 2008 Apr; 298(4):56-63.

Solomon D E (2002) An in vitro examination of an ECM matrix for use in wound healing. Int. J. Exp. Path. 83, 209-216.

Solomon D E (2006) UK patent application, GB 2442271A ‘An in vitro human skin assay test protocol which directly uses the natural cellular interactions between un-manipulated autologous human epidermal and dermal cells’. Solomon D E (2007) UK patent application, GB 2448142A ‘Recognition of melanocytes in benign and cancerous tissue’.

McHeik J N, Barrault C, Vincent G, Grammatico F, Peci S, Garnier J, Bernard F X, Deguercy A, Levard G. (2009) Cultured keratinocyte cells from foreskin and future application for burns in children. Ann Chir Plast Esthet., 2009 Dec; 54(6): 528-32. doi:10.1016/j.anplas.2008.10.016, PMID: 19195754.

Duncan C O, Shelton R M, Naysaria Balderson D S, Papini R P, Barralet J E. (2005) In vitro transfer of keratinocytes: comparison of transfer from fibrin membrane and delivery by aerosol spray. J Biomed Mater Res B Appl Biomater. May: 73(2):221-8.

Currie L J, Martin R, Sharpe J R, James S E. (2003) A comparison of keratinocyte cell sprays with and without fibrin glue. Burns. 29(7):677-85.

Barton S P and Marks R. (1981) Changes in emulsions of keratinocytes due to trypsin. Arch Dermatol Res 271:245-257.

Bishop L A, Kee W J, Zhu A J, Watt F M. (1998) Lack of intrinsic polarity in the ligand-binding ability of keratinocyte betal integrins. Exp Dermatol. Dec: 7(6):350-61.

Spichkina O G, Kalmykova N V, Kukhareva L V, Voronkina I V, Blinova M I, Pinaev G P.(2006) Isolation of human basal keratinocytes by selective adhesion to extracellular matrix proteins.] Tsitologiia. 48 (10):841-7. Pubmed/17162842.

1) A topical ‘light emulsion’ First Aid spray, which may or may not contain antibiotics, for wounds and burns with pain relieving properties.

2) The cornstarch granule is a cell scaffold and used with antibiotic(s) may be topically applied to aid healing in soft tissue repair, for example, after Mohs micrographic surgery for skin cancer including basal/squamous cell carcinomas.

3) For deep, wider wounds or for cosmetic repair, without the use of the aerosol propellant the nutritional cell scaffold and antibiotic(s) are first applied followed by an autologous mixture of human papillary and reticular dermal fibroblasts followed by autologous epidermal brown rosettes isolated as described in this specification; procedure is repeated as required for guided tissue regeneration.
